(19) United States
(12) Patent Application Publication
(10) Pub. No.: US 2018/0177778 A1
(43) Pub. Date: Jun. 28, 2018

(54) Methods for Treating HCV

(71) Applicant: AbbVie Inc., North Chicago, IL (US)

(72) Inventors: Walid M. Awni, Green Oaks, IL (US); Tolga Baykal, Libertyville, IL (US); Barry M. Bernstein, Mequon, WI (US); Scott c. Brun, Green Oaks, IL (US); Daniel E. Cohen, Wilmette, IL (US); Emily O. Dumas, Libertyville, IL (US); Sandeep Dutta, Lincolnshire, IL (US); Amit Khatri, Waukegan, IL (US); Cheri E. Klein, Northbrook, IL (US); Rajeev M. Menon, Buffalo Grove, IL (US); Sven Mensing, Ilversheim (DE); Thomas J. Podsadecki, Chicago, IL (US); Lino X Rodrigues Jr., Chicago, IL (US); Regis A. Vilchez, Lake Forest, IL (US)

(73) Assignee: AbbVie Inc., North Chicago, IL (US)

(21) Appl. No.: 15/738,762

(22) PCT Filed: Jun. 28, 2016
(86) PCT No.: PCT/US2016/039838

§ 371 Date: Dec. 21, 2017

Related U.S. Application Data
(60) Provisional application No. 62/267,623, filed on Dec. 15, 2015.
(60) Provisional application No. 62/186,185, filed on Jun. 29, 2015.

(51) Int. Cl. A61K 31/497; A61P 31/14; A61K 31/4025; A61K 31/427; A61K 31/7056

(52) U.S. Cl. CPC A61K 31/497; A61P 31/14; A61K 31/7056; A61K 31/427; A61K 31/4025

(57) ABSTRACT

The present invention features interferon-free therapies for treating Hepatitis C Virus (HCV) genotypes 1b, 2, 3 or 4. In one aspect, the therapies comprises administering Compound 1 (Paritaprevir), Ritonavir, and Compound 2 (Ombitasvir) to a subject infected with HCV genotype 1b or 4, wherein the therapies do not include the administration of any interferon, and the therapies last from 8 to 12 weeks. Preferably, the therapies do not include the administration of any ribavirin.

### FIELD OF THE INVENTION

The present invention relates to interferon-free treatment for HCV.

### BACKGROUND OF THE INVENTION

The hepatitis C virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

Chronic HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often incomplete. Therefore, there is a need for new therapies to treat HCV infection.

### BRIEF DESCRIPTION OF THE DRAWINGS

FIG. 1 shows the predicted median and 90% confidence interval of sustained virological response (SVR) percentage for different treatment durations of a 2-DAA regimen without ribavirin; wherein the 2 DAAs include (i) Compound 1 with ritonavir (Compound 1/r) and (ii) Compound 2.

### DESCRIPTION OF THE INVENTION

The present invention feature methods of treatment for HCV genotype (GT) 1b, 2, 3 or 4. The treatment comprises administering Compound 1 (paritaprevir) or a pharmaceutically acceptable salt thereof, and Compound 2 (ombitasvir) or a pharmaceutically acceptable salt thereof, to a patient infected with HCV genotype 1b, 2, 3, or 4. The treatment does not include administration of any interferon. To improve pharmacokinetics, Compound 1 or the salt thereof preferably is co-administered with ritonavir or another CYP3A4 inhibitor (e.g., cobicistat).

A treatment regimen of the invention generally constitutes a complete treatment, and no subsequent interferon-containing regimen is intended. Therefore, a treatment or use described herein generally does not include any subsequent interferon-containing treatment.

A treatment regimen of the invention preferably lasts no more than 12 weeks. More preferably, a treatment regimen of the invention lasts from 8 to 12 weeks, such as 8, 9, 10, 11, or 12 weeks. Highly preferably, a treatment regimen of the invention lasts for 12 weeks.

Compound 1 (paritaprevir,

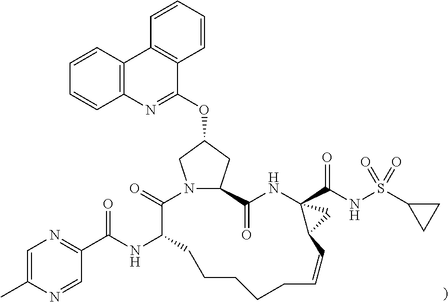 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

is also known as (2R,6S,13aS,14aR,16aS,Z)—N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide. Compound 1 is a potent HCV protease inhibitor. The synthesis and formulation of Compound 1 are described in U.S. Patent Application Publication Nos. 2010/0144608 and 2011/0312973, both of which incorporated herein by reference in their entireties. The generic name for Compound 1 is paritaprevir.

Compound 2 (ombitasvir,

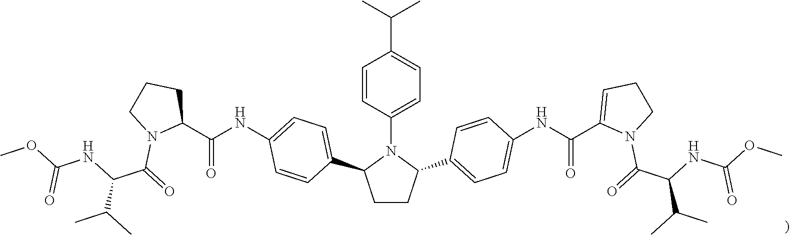

is also known as dimethyl (2S,2′S)-1,1′-((2S,2′S)-2,2′-(4,4′-((2S,5S)-1-(4-tert-butylphenyl)pyrrolidine-2,5,diyl)bis(4,1-phenylene))bis(azanediyl)bis(oxomethylene)bis(pyrrolidine-2,1-diyl)bis(3-methyl-1-oxobutane-2,1-diyl)dicarbamate. The preparation and formulation of Compound 2 are described in U.S. Patent Application Publication Nos. 2010/0317568 and 2012/0258909, both of which are incorporated herein by reference in their entireties. The generic name for Compound 2 is ombitasvir.

In any method or treatment regimen of the invention, or any aspect, embodiment or example described herein, Compound 1 can be administered, for example, 100 mg once daily (QD), Compound 2 25 mg QD, and ritonavir 100 mg QD.

In any method or treatment regimen of the invention, or any aspect, embodiment or example described herein, Compound 1, ritonavir and Compound 2 can be, for example, co-formulated in a single dosage form. Preferably, Compound 1, ritonavir and Compound 2 are co-formulated in a single solid dosage form. More preferably, Compound 1, ritonavir and Compound 2 are each formulated in an amorphous solid dispersion comprising a hydrophilic polymer and a pharmaceutically acceptable surfactant. Compound 1, ritonavir and Compound 2 can be formulated in the same solid dispersion; Compound 1, ritonavir and Compound 2 can also be formulated in separate solid dispersions and then mixed together to provide a single solid dosage form.

In any method or treatment regimen of the invention, or any aspect, embodiment or example described herein, Compound 1, ritonavir and Compound 2 can be, for example, co-formulated in a single dosage form which comprises 75 mg Compound 1, 50 mg ritonavir, and 12.5 mg Compound 2.

In any method or treatment regimen of the invention, or any aspect, embodiment or example described herein, a treatment regimen of the invention can, for example, further comprise administering ribavirin to the patient. Preferably, in any method or treatment regimen of the invention, or any aspect, embodiment or example described herein, a treatment regimen of the invention does not include administration of any ribavirin.

In any method or treatment regimen of the invention, or any aspect, embodiment or example described herein, the patient can be a treatment-naïve patient, an interferon null responder, or an interferon non-responder.

In any method or treatment regimen of the invention, or any aspect, embodiment or example described herein, the patient can be a treatment-experienced patient (e.g., an interferon null responder or an interferon non-responder).

In any method or treatment regimen of the invention, or any aspect, embodiment or example described herein, the patient can be a non-cirrhotic, treatment-naïve patient.

In any method or treatment regimen of the invention, or any aspect, embodiment or example described herein, the patient can be a non-cirrhotic, treatment-experienced patient (e.g., an interferon null responder or an interferon non-responder).

In any method or treatment regimen of the invention, or any aspect, embodiment or example described herein, the patient can be a treatment-naïve patient with compensated cirrhosis.

In any method or treatment regimen of the invention, or any aspect, embodiment or example described herein, the patient can be a treatment-experienced patient (e.g., an interferon null responder or an interferon non-responder) with compensated cirrhosis.

In any method or treatment regimen of the invention, or any aspect, embodiment or example described herein, the patient can be an interferon null responder with compensated cirrhosis.

In any method or treatment regimen of the invention, or any aspect, embodiment or example described herein, the patient can be an interferon non-responder with compensated cirrhosis.

In any method or treatment regimen of the invention, or any aspect, embodiment or example described herein, the patient can be a patient without cirrhosis.

In any method or treatment regimen of the invention, or any aspect, embodiment or example described herein, the patient can be a cirrhotic patient.

In any method or treatment regimen of the invention, or any aspect, embodiment or example described herein, the patient can be a patient with compensated cirrhosis.

In any method or treatment regimen of the invention, or any aspect, embodiment or example described herein, Compound 1/r and Compound 2 can also be used in combination with Compound 3 (N-(6-(3-tert-butyl-5-(2,4-dioxo-3,4-dihydropyrimidin-1(2H)-yl)-2-methoxyphenyl)naphthalen-2-yl)methanesulfonamide), also known as dasabuvir, as described below.

In any method or treatment regimen of the invention, or any aspect, embodiment or example described herein, Compound 1/r and Compound 2 can be administered QD.

In any method or treatment regimen of the invention, or any aspect, embodiment or example described herein, Compound 1/r and Compound 2 can be administered QD; and if Compound 3 (dasabuvir) is also administered, Compound 3 can be administered BID.

In any method or treatment regimen of the invention, or any aspect, embodiment or example described herein, Compound 1/r and Compound 2 can be administered QD; and if Compound 3 is also administered, Compound 3 can be administered QD.

In any method or treatment regimen of the invention, or any aspect, embodiment or example described herein, the patient can be a patient infected with HCV GT 1.

In any method or treatment regimen of the invention, or any aspect, embodiment or example described herein, the patient can be a patient infected with HCV GT 1a.

In any method or treatment regimen of the invention, or any aspect, embodiment or example described herein, the patient can be a patient infected with HCV GT 1b.

In any method or treatment regimen of the invention, or any aspect, embodiment or example described herein, the patient can be a patient infected with HCV GT 4.

In any method or treatment regimen of the invention, or any aspect, embodiment or example described herein, the patient can be a patient infected with HCV GT 1 and without cirrhosis.

In any method or treatment regimen of the invention, or any aspect, embodiment or example described herein, the patient can be a patient infected with HCV GT 1a and without cirrhosis.

In any method or treatment regimen of the invention, or any aspect, embodiment or example described herein, the patient can be a patient infected with HCV GT 1b and without cirrhosis.

In any method or treatment regimen of the invention, or any aspect, embodiment or example described herein, the patient can be a patient infected with HCV GT 4 and without cirrhosis.

In any method or treatment regimen of the invention, or any aspect, embodiment or example described herein, the patient can be a patient infected with HCV GT 1 and with compensated cirrhosis.

In any method or treatment regimen of the invention, or any aspect, embodiment or example described herein, the patient can be a patient infected with HCV GT 1a and with compensated cirrhosis.

In any method or treatment regimen of the invention, or any aspect, embodiment or example described herein, the patient can be a patient infected with HCV GT 1b and with compensated cirrhosis.

In any method or treatment regimen of the invention, or any aspect, embodiment or example described herein, the patient can be a patient infected with HCV GT 4 and with compensated cirrhosis.

In one aspect, the present invention features methods of treatment for HCV genotype 1b. The treatment comprises administering Compound 1 or a pharmaceutically acceptable salt thereof, and Compound 2 or a pharmaceutically acceptable salt thereof, to a patient infected with HCV genotype 1b, wherein the treatment does not include administration of interferon to the patient. The treatment can last from 8 to 12 weeks. For example, the treatment can last for 8, 9, 10, 11 or 12 weeks. Preferably, the treatment lasts for 12 weeks.

Compound 1 preferably is co-administered with ritonavir. Another CYP3A4 inhibitor, such as cobicistat, can also be used in lieu of ritonavir.

In any method or treatment regimen of this aspect of the invention, the patient being treated can be a treatment-naïve patient.

In any method or treatment regimen of this aspect of the invention, the patient being treated can be a treatment-experienced patient

In any method or treatment regimen of this aspect of the invention, the patient being treated can be an interferon null responder.

In any method or treatment regimen of this aspect of the invention, the patient being treated can be an interferon non-responder.

In any method or treatment regimen of this aspect of the invention, the patient being treated can be a non-cirrhotic, treatment-naïve patient.

In any method or treatment regimen of this aspect of the invention, the patient being treated can be a non-cirrhotic, treatment-experienced patient

In any method or treatment regimen of this aspect of the invention, the patient being treated can be a non-cirrhotic, interferon null responder.

In any method or treatment regimen of this aspect of the invention, the patient being treated can be a non-cirrhotic, interferon non-responder.

In any method or treatment regimen of this aspect of the invention, the patient being treated can be a treatment-naïve patient with compensated cirrhosis.

In any method or treatment regimen of this aspect of the invention, the patient being treated can be a treatment-experienced patient with compensated cirrhosis.

In any method or treatment regimen of this aspect of the invention, the patient being treated can be an interferon null responder with compensated cirrhosis.

In any method or treatment regimen of this aspect of the invention, the patient being treated can be an interferon non-responder with compensated cirrhosis.

In any method or treatment regimen of this aspect of the invention, the patient can be a patient without cirrhosis.

In any method or treatment regimen of this aspect of the invention, the patient can be a cirrhotic patient.

In any method or treatment regimen of this aspect of the invention, the patient can be a patient with compensated cirrhosis

In this aspect of invention or any embodiment or example thereof, a treatment regimen can further comprise administering ribavirin to said patient. Preferably, in this aspect of invention or any embodiment or example thereof, a treatment regimen does not comprise administration of any ribavirin to said patient.

In another aspect, the present invention features methods of treatment for HCV genotype 4. The treatment comprises administering Compound 1 or a pharmaceutically acceptable salt thereof, and Compound 2 or a pharmaceutically acceptable salt thereof, to a patient infected with HCV genotype 4, wherein the treatment does not include administration of any interferon to the patient. The treatment can last from 8 to 12 weeks. For example, the treatment can last for 8, 9, 10, 11 or 12 weeks. Preferably, the treatment lasts for 12 weeks.

Compound 1 preferably is co-administered with ritonavir. Another CYP3A4 inhibitor, such as cobicistat, can also be used in lieu of ritonavir.

In any method or treatment regimen of this aspect of the invention, the patient being treated can be a treatment-naïve patient.

In any method or treatment regimen of this aspect of the invention, the patient being treated can be a treatment-experienced patient

In any method or treatment regimen of this aspect of the invention, the patient being treated can be an interferon null responder.

In any method or treatment regimen of this aspect of the invention, the patient being treated can be an interferon non-responder.

In any method or treatment regimen of this aspect of the invention, the patient being treated can be a non-cirrhotic, treatment-naïve patient.

In any method or treatment regimen of this aspect of the invention, the patient being treated can be a non-cirrhotic, treatment-experienced patient

In any method or treatment regimen of this aspect of the invention, the patient being treated can be a non-cirrhotic, interferon null responder.

In any method or treatment regimen of this aspect of the invention, the patient being treated can be a non-cirrhotic, interferon non-responder.

In any method or treatment regimen of this aspect of the invention, the patient being treated can be a treatment-naïve patient with compensated cirrhosis.

In any method or treatment regimen of this aspect of the invention, the patient being treated can be a treatment-experienced patient with compensated cirrhosis.

In any method or treatment regimen of this aspect of the invention, the patient being treated can be an interferon null responder with compensated cirrhosis.

In any method or treatment regimen of this aspect of the invention, the patient being treated can be an interferon non-responder with compensated cirrhosis.

In any method or treatment regimen of this aspect of the invention, the patient can be a patient without cirrhosis.

In any method or treatment regimen of this aspect of the invention, the patient can be a cirrhotic patient.

In any method or treatment regimen of this aspect of the invention, the patient can be a patient with compensated cirrhosis

Preferably, in this aspect of invention or any embodiment or example thereof, a treatment regimen comprises administering ribavirin to said patient. Alternatively, in this aspect of invention or any embodiment or example thereof, a treatment regimen does not include administration of any ribavirin to said patient.

As used herein, non-limiting examples of interferon include pegylated interferon (pegIFN), such as pegylated interferon-alpha-2a or pegylated interferon-alpha-2b. Specific examples of interferon include, but are not limited to, Pegasys, PegIntron, Roferon A, or Intron A. Specific examples of ribavirin (RBV) include, but are not limited to, Copegus, Rebetol, or Ribasphere.

GUIDANCE FOR INDUSTRY—CHRONIC HEPATITIS C VIRUS INFECTION: DEVELOPING DIRECT-ACTING ANTIVIRAL AGENTS FOR TREATMENT (FDA, September 2010, draft guidance) define treatment-naïve, partial responder, responder relapser (i.e., rebound), and null responder patients. The interferon non-responder patients include null responder, partial responder as well as rebound patients.

Various measures can be used to evaluate the responsiveness or effectiveness of an HCV treatment. One such measure is rapid virologic response (RVR), meaning that HCV is undetectable in the subject after 4 weeks of treatment. Another measure is early virologic response (EVR), meaning that the subject has >2 log₁₀ reduction in viral load after 12 weeks of treatment. Another measure is complete EVR (cEVR), meaning the HCV is undetectable in the serum of the subject after 12 weeks of treatment. Another measure is extended RVR (eRVR), meaning achievement of both RVR and cEVR, that is, HCV is undetectable at week 4 and 12. Another measure is the presence or absence of detectable virus at the end of therapy (EOTR). Another measure is SVR, which, as used herein, means that the virus is undetectable at the end of therapy and for at least 8 weeks after the end of therapy (SVR8); preferably, the virus is undetectable at the end of therapy and for at least 12 weeks after the end of therapy (SVR12); more preferably, the virus is undetectable at the end of therapy and for at least 16 weeks after the end of therapy (SVR16); and highly preferably, the virus is undetectable at the end of therapy and for at least 24 weeks after the end of therapy (SVR24). A desired treatment should achieve significantly high SVR rates.

Preferably, a treatment regimen of the invention achieves at least 80% SVR12 rate. More preferably, a treatment regimen of the invention achieves at least 90% SVR12 rate. Highly preferably, a treatment regimen of the invention achieves at least 95% SVR12 rate.

A treatment regimen of the invention may also comprise administering to the patient one or more other HCV direct acting agents (DAAs), such as other HCV protease inhibitors, HCV polymerase inhibitors, other HCV NS5A inhibitors, cyclophilin inhibitors, or combinations thereof.

Non-limiting examples of HCV protease inhibitors include telaprevir (Vertex), boceprevir (Merck), BI-201335 (Boehringer Ingelheim), GS-9451 (Gilead), and BMS-650032 (BMS). Other suitable protease inhibitors include, but are not limited to, ACH-1095 (Achillion), ACH-1625 (Achillion), ACH-2684 (Achillion), AVL-181 (Avila), AVL-192 (Avila), BMS-650032 (BMS), danoprevir (RG7227/ITMN-191, Roche), GS-9132 (Gilead), GS-9256 (Gilead), IDX-136 (Idenix), IDX-316 (Idenix), IDX-320 (Idenix), MK-5172 (Merck), narlaprevir (Schering-Plough Corp), PHX-1766 (Phenomix), TMC-435 (Tibotec), vaniprevir (MK-7009, Merck), VBY708 (Virobay), VX-500 (Vertex), VX-813 (Vertex), and VX-985 (Vertex).

Non-limiting examples of non-nucleoside HCV polymerase inhibitors include GS-9190 (Gilead), BI-207127 (Boehringer Ingelheim), and VX-222 (VCH-222) (Vertex & ViraChem). Non-limiting examples of nucleotide HCV polymerase inhibitors include GS-7977 (Gilead). Other suitable, non-limiting examples of HCV polymerase inhibitors include ANA-598 (Anadys), BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-759 (Vertex), GS-6620 (Gilead), IDX-102 (Idenix), IDX-184 (Idenix), INX-189 (Inhibitex), MK-0608 (Merck), RG7128 (Roche), TMC64912 (Medivir), GSK625433 (GlaxoSmithKline), BCX-4678 (BioCryst), ALS-2200 (Alios BioPharma/Vertex), and ALS-2158 (Alios BioPharma/Vertex).

Non-limiting examples of NS5A inhibitors include BMS-790052 (BMS) and GS-5885 (Gilead). Other non-limiting examples of suitable NS5A inhibitors include GSK62336805 (GlaxoSmithKline), ACH-2928 (Achillion), AZD2836 (Astra-Zeneca), AZD7295 (Astra-Zeneca), BMS-790052 (BMS), BMS-824393 (BMS), GS-5885 (Gilead), PPI-1301 (Presidio), PPI-461 (Presidio) A-831 (Arrow Therapeutics), and A-689 (Arrow Therapeutics).

Non-limiting examples of cyclophilin inhibitors include alisporovir (Novartis & Debiopharm), NM-811 (Novartis), and SCY-635 (Scynexis).

Compound 1 (or a pharmaceutically acceptable salt thereof) and Compound 2 (or a pharmaceutically acceptable salt thereof) can be used to treat HCV patients with cirrhosis. The patients can infected with HCV genotypes 1, 2, 3, 4, 5 or 6, such as genotype 1a or 1b, and the cirrhosis can be either compensated or decompensated. The methods comprise administering Compound 1 or a pharmaceutically acceptable salt thereof, and Compound 2 or a pharmaceutically acceptable salt thereof, to such a patient, wherein the treatment does not include administration of interferon to the patient. The treatment can last from 8 to 12 weeks; for example, the treatment can last for 8, 9, 10, 11 or 12 weeks. Preferably, the treatment lasts for 12 weeks. Longer treatment durations can also be used, such as 24 weeks or a less duration. Ribavirin can be administered; or alternatively, the treatment does not include administering ribavirin. Preferably, the treatment further comprises administering ribavirin and N-(6-(3-tert-butyl-5-(2,4-dioxo-3,4-dihydropyrimidin-1(2H)-yl)-2-methoxyphenyl)naphthalen-2-yl)methanesulfonamide (or a pharmaceutically acceptable salt thereof). See U.S. Patent Application Publication No. 2013/0102525. To improve pharmacokinetics, Compound 1 or the salt thereof preferably is co-administered with ritonavir or another CYP3A4 inhibitor (e.g., cobicistat). Other known DAA combinations that are currently being tested in clinical trials can also be used to treat cirrhotic patients in similar regimens.

In any aspect, embodiment, preference, example, method or treatment regimen described herein, the patient being treated can be a pediatric patient, and the dosing of Compound 1, Compound 2 Compound 3 and ritonavir can follow the following schedule: (1) for a pediatric patient with a weight of up to 14 kg, 35 mg Compound 1, 25 mg ritonavir, and 5 mg Compound 2 once daily and, if needed, 50 mg Compound 3 twice daily; (2) for a pediatric patient with a weight of from 15 to 29 kg, 50 mg Compound 1, 35 mg ritonavir, and 10 mg Compound 2 once daily and, if needed, 100 mg Compound 3 twice daily; (3) for a pediatric patient with a weight of from 30 to 44 kg, 100 mg Compound 1, 70 mg ritonavir, and 15 mg Compound 2 once daily and, if needed, 150 mg Compound 3 twice daily; (4) for a pediatric patient with a weight of 45 kg or greater, 150 mg Compound 1, 100 mg ritonavir, and 25 mg Compound 2 once daily and, if needed, 250 mg Compound 3 twice daily.

In any aspect, embodiment, preference, example, method or treatment regimen described herein, the patient being treated can be a pediatric patient, and the dosing of Compound 1, Compound 2 Compound 3 and ritonavir can follow the following schedule: (1) for a pediatric patient with an age of 3-8 years old and a weight of up to 14 kg, 35 mg Compound 1, 25 mg ritonavir, and 5 mg Compound 2 once daily and, if needed, 50 mg Compound 3 twice daily; (2) for a pediatric patient with an age of 3-8 years old and a weight of from 15 to 29 kg, 50 mg Compound 1, 35 mg ritonavir, and 10 mg Compound 2 once daily and, if needed, 100 mg Compound 3 twice daily; (3) for a pediatric patient with an age of 9-11 years old and a weight of from 15 to 29 kg, 60 mg Compound 1, 40 mg ritonavir, and 10 mg Compound 2 once daily and, if needed, 100 mg Compound 3 twice daily; (4) for a pediatric patient with an age of 3-8 years old and a weight of from 30 to 44 kg, 100 mg Compound 1, 70 mg ritonavir, and 15 mg Compound 2 once daily and, if needed, 150 mg Compound 3 twice daily; (5) for a pediatric patient with an age of 9-11 years old and a weight of from 30 to 44 kg, 90 mg Compound 1, 60 mg ritonavir, and 15 mg Compound 2 once daily and, if needed, 150 mg Compound 3 twice daily; (6) for a pediatric patient with an age of 12-18 years old and a weight of from 30 to 44 kg, 80 mg Compound 1, 55 mg ritonavir, and 12.5 mg Compound 2 once daily and, if needed, 125 mg Compound 3 twice daily; (7) for a pediatric patient with a weight of 45 kg or greater, regardless of age, 150 mg Compound 1, 100 mg ritonavir, and 25 mg Compound 2 once daily and, if needed, 250 mg Compound 3 twice daily.

It should be understood that the above-described embodiments and the following examples are given by way of illustration, not limitation. Various changes and modifications within the scope of the present invention will become apparent to those skilled in the art from the present description.

### Example 1. Interferon- and Ribavirin-Free Treatment of HCV Genotype 1b

Treatment-naïve patients and prior pegIFN/RBV null responders received Compound 1 (150 mg QD), ritonavir (100 mg QD) and Compound 2 (25 mg QD) for 12 weeks. 42 treatment-naïve patients and 40 prior pegIFN/RBV null responders with chronic HCV genotype 1b infection were enrolled. All patients are non-cirrhotic. Baseline characteristics are shown in Table 1. Observed rates of HCV RNA <25 IU/mL (detection limit) at treatment weeks 4 and 12 of the treatment, as well as observed SVR₄ rates (percent of patients with HCV RNA <25 IU/mL at post-treatment week 4) are summarized in Table 1. SVR₄ rate was 100% among treatment-naïve patients and 87.9% among prior null responders.

Further follow-up showed that among the 39 treatment-naïve patients that were actually tested at post-treatment week 8, 100% of the patients did not have detectable HCV RNA; and among the 30 treatment-naïve patients that were actually tested at post-treatment week 12, 97% of the patients (29/30) did not have detectable HCV RNA. Follow-up testing showed that among the 42 treatment-naïve patients, 40 patients achieved SVR₁₂, and the two remaining patients did not achieve SVR₁₂ due to loss to follow-up.

Testing also showed that among the 39 null responders that were actually tested at post-treatment week 4, 90% of the patients (35/39) did not have detectable HCV RNA. Further testing at post-treatment week 8 showed that 87% of the null responders that were actually tested (26/30) did not have detectable HCV RNA. Follow-up testing showed that among the 40 prior pegIFN/RBV null responders, 36 patients achieved SVR₁₂.

Among the 82 patients, there were no discontinuations due to adverse events (AE) or laboratory abnormalities. There were 2 serious AEs (both not related to study drug). Two subjects interrupted study drug due to AEs. One interruption was probably related to study drug (increased ALT, AST, and bilirubin); these values improved during resumed treatment or after completion.

TABLE 1

| | |
|---|---|
| Treatment-naïve | |
| Patients | Prior Null Responders |
| (N = 42) | (N = 40) |

Baseline characteristics

| | | | | |
|---|---|---|---|---|
| Male, n (%) | 25 | (59.5) | 15 | (37.5) |
| White race, n (%) | 27 | (65.9) | 39 | (97.5) |
| Age <50 yr, n (%) | 7 | (16.7) | 13 | (32.5) |
| Weight <85 kg, n (%) | 27 | (64.3) | 28 | (70.0) |
| IL28B CC, n (%) | 13 | (31.7) | 2 | (5.0) |

Efficacy

| | | | | |
|---|---|---|---|---|
| HCV RNA <25 IU/mL at | 42/42 | (100) | 39/40 | (97.5) |
| treatment week 4, n/N (%)* | | | | |
| HCV RNA <25 IU/mL at | 40/40 | (100) | 39/40 | (97.5) |
| treatment week 12, n/N (%)* | | | | |
| SVR₄, n/N (%)* | 39/39 | (100) | 29/33 | (87.9) |

| | | |
|---|---|---|
| On-treatment failure, n | 0 | 1 |
| Relapse, n | 0 | 3 |

*Observed data. Excludes patients with data missing for reasons besides virologic failure

### Example 2. Clinical Modeling for Interferon-Free Treatment of HCV Genotype 4

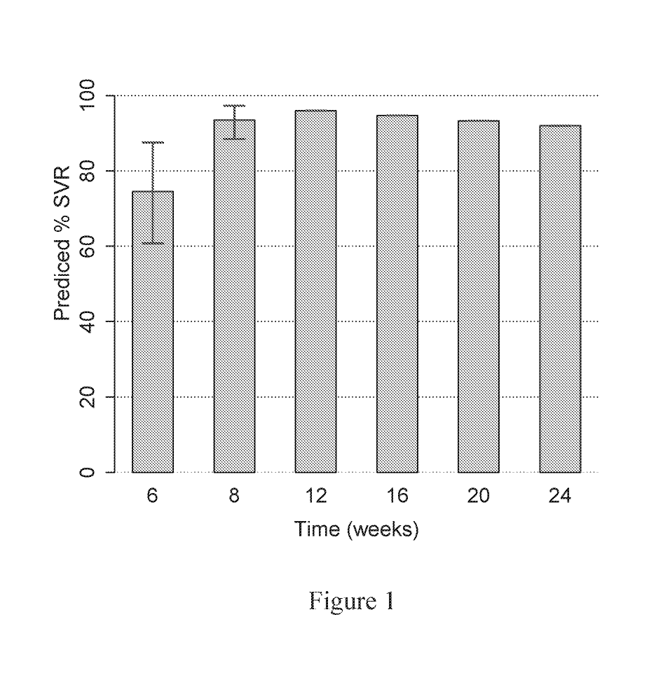

A novel clinical model for evaluating appropriate doses and durations of interferon-free HCV therapies using combinations of DAAs has been described in Example 6 of U.S. Patent Application Publication No. 2013/0102525, which example is incorporated herein by reference. Data from clinical studies, as well as in vitro replicon experiments, of Compound 1 and Compound 2 were used for estimating the pharmacokinetic and viral dynamic model parameters. In vivo parameters for genotype 4 were approximated using in vitro data, based on the relationship between the in vivo and in vitro data for genotype 1. The model predicts that following 8 or 12 weeks of dosing with the combination of Compound 1 (150 mg QD), ritonavir (100 mg QD) and Compound 2 (25 mg QD), over 90% of genotype 4 treatment-naïve patients can achieve SVR. See FIG. 1. FIG. 1 shows the predicted median SVR percentage (“% SVR”) and 90% confidence interval (the vertical bar at the top of each SVR percentage column) for different treatment durations using a combination of Compound 1, ritonavir and Compound 2, without interferon. Similar or better SVR rates are expected when ribavirin is included in the regimen.

### Example 3. Clinical Study of Interferon-Free Treatment of HCV Genotype 4

A clinical study of interferon-free treatment of HCV genotype 4 was conducted. Two groups of treatment naïve patients with HCV GT 4 infection were enrolled in the study, each group including about 40 patients. Compound 1 (150 mg QD), ritonavir (100 mg QD), and Compound 2 (25 mg QD) were administered to each patient in both groups. Weight-based Ribavirin was also administered to the patients in the first group, but not to the second group. The baseline characteristics of these patients are summarized in Table 2.

After 12-week treatment, the first group of patients (with ribavirin) achieved about 100% SVR12 rate, and the second group (without ribavirin) achieved about 90% SVR12.

TABLE 2

| | | | Treatment-naive Patients | |
|---|---|---|---|---|
| | Treatment-naïve Patients | | (Compound 1/ritonavir + | |
| | (Compound 1/ritonavir + | | Compound 2 + | |
| | Compound 2) | | Ribavirin) | |
| | (N = 44) | | (N = 42) | |
| Male, n (%) | 24 | (54.5) | 27 | (64.3) |
| White race, n (%) | 37 | (84.1) | 38 | (90.5) |
| IL28B CC, n (%) | 12 | (27.3) | 11 | (26.2) |
| Fibrosis stage, ≥F2, | 5 | (11.6)* | 9 | (21.4) |
| n (%) | | | | |
| Baseline HCV RNA | 6.07 | (0.62) | 6.12 | (0.58) |
| level, log₁₀ IU/mL, | | | | |
| mean (SD) | | | | |
| RVR, n/N (%) | 43/43 | (100) | 41/42 | (97.6)** |
| EOTR, n/N (%) | 42/43 | (97.7) | 42/42 | (100) |

| | | |
|---|---|---|
| Breakthrough | 1 | 0 |

*Fibrosis score was missing for one patient in this group.
**One patient did not have HCV RNA suppressed below 25 IU/mL until Week 6. This patient did not achieve RVR, but achieved EOTR.

In another arm, 49 interferon partial/null responders or relapsers with HCV GT 4 infection were enrolled and treated with Compound 1 (150 mg QD), ritonavir (100 mg QD), Compound 2 (25 mg QD) and ribavirin for 12 weeks. The SVR4 for this group of patients was 100%. Seven (7) of the 49 patients were tested at post-treatment week 12, and the SVR12 was 100%. Further testing showed that all 49 patients in this arm achieved SVR12 (100%).

Further analysis showed that Compound 1/ritonavir+Compound 2, either with or without ribavirin, achieved high SVR rate among patients with different GT 4 subtypes. Accordingly, in any method or treatment regimen of the invention for treating GT 4, or any aspect, embodiment or example described herein for treating GT 4, identification of specific GT4 subtype prior to the initiation of therapy is optional. For example, in any method or treatment regimen of the invention for treating GT 4, or any aspect, embodiment or example described herein for treating GT 4, the method preferably does not comprise the identification of specific GT4 subtype prior to the initiation of therapy.

### Example 4. Clinical Study of Interferon-Free Treatment of HCV Genotype 1b

This study was a double-blind controlled trial. Subjects were randomized (1:1) to 12 weeks of treatment with Compound 1 (150 mg QD), ritonavir (100 mg QD), Compound 2 (25 mg QD), and Compound 3 (250 mg BID), with weight-based ribavirin (1000 mg or 1200 mg daily divided BID, Arm A) or placebo for ribavirin (Arm B). Compound 3 (dasabuvir) is N-(6-(3-tert-butyl-5-(2,4-dioxo-3,4-dihydropyrimidin-1(2H)-yl)-2-methoxyphenyl)naphthalen-2-yl)methanesulfonamide

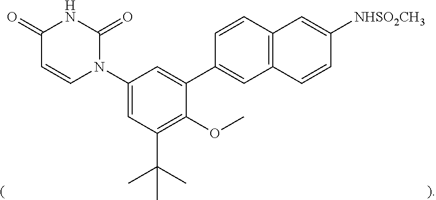

See International Application Publication No. WO2009/039127.

419 subjects received the above regimen, baseline characteristics as shown in Table 3. These subjects were infected with HCV GT 1b, and were treatment-naïve and non-cirrhotic. SVR12 rates (intent-to-treat) were 99.5% (Arm A) and 99.0% (Arm B) with no on-treatment virologic failure or post-treatment relapse among subjects receiving the above regimen without ribavirin. 19 subjects in Arm A and 0 in Arm B (P<0.001) had hemoglobin <10 g/dL. The most common adverse events in Arms A and B were headache (24.3% vs. 23.4%, P=NS) and fatigue (21.4% vs. 23.0%, P=NS.) No subjects discontinued due to adverse events.

TABLE 3

| | Arm A | | Arm B | |
|---|---|---|---|---|
| | (with RBV) | | (without RBV) | |
| | N = 210 | | N = 209 | |
| Male, n (%) | 106 | (50.5) | 86 | (41.1) |
| White race, n (%) | 198 | (94.3) | 196 | (94.2) |
| Age, mean (SD) | 48.4 | (11.9) | 49.2 | (12.0) |
| IL28B CC, n (%) | 44 | (21.0) | 44 | (21.1) |
| Baseline HCV RNA, log₁₀ IU/mL, | 6.29 | (0.77) | 6.33 | (0.67) |
| mean (SD) | | | | |
| SVR_{12,} n (%) | 209 | (99.5) | 207 | (99.0) |

| | | | |
|---|---|---|---|
| On-treatment virologic failure | 1 | (0.5) | 0 |

| Relapse by post-treatment Week 12 | 0 | 0 | |
|---|---|---|---|
| Missing SVR₁₂ data | 0 | 2 | (1.0) |

This study shows that the combination of Compound 1/r, Compound 2 and Compound 3 is highly efficacious and safe with or without RBV for the treatment of HCV GT-1b infection. Both regimens were noninferior and superior compared to the historical rate for telaprevir+pegIFN/RBV. The addition of RBV appears not to provide additional clinical benefit for this GT-1b population when treated with Compound 1/r, Compound 2 and Compound 3

### Example 5. Clinical Study of Interferon-Free Treatment of HCV Genotype 1b

This example describes a phase 3 open-label study in HCV GT1b-infected patients who were randomized 1:1 to receive Compound 1 (150 mg QD) dosed with ritonavir (100 mg QD), Compound 2 (25 mg QD), and Compound 3 (250 mg BID) with RBV (Arm A) or without RBV (Arm B) for 12 weeks. 12-week post-treatment SVR rates (SVR12) for each treatment arm were compared to a historical telaprevir plus pegIFN/RBV threshold. Adverse events (AEs) were recorded for all patients receiving at least 1 dose of study drug. All patients were non-cirrhotic.

Of 187 treatment-experienced, randomized GT1b-infected patients, 186 were dosed with study drug and included in safety analyses; 179 patients received Compound 1/r and Compound 2 co-formulated drug and were included in intent-to-treat (ITT) efficacy analyses. In the ITT population, 35.2% were null-responders, 28.5% partial responders, and 36.3% relapsers to previous pegIFN/RBV treatment. Mean age (54.2 vs. 54.2 years), sex (49.5% vs. 60.0% male), and IL28B genotype CC (11.0% vs. 7.4%) were comparable between Arms A and B, respectively. After 12 weeks of treatment, intent-to-treat SVR₁₂ rates were 96.6% for Arm A and 100% for Arm B (Table 4). Similarly high SVR12 rates were observed in null-responders, partial responders, and relapsers. No patients experienced virologic failure; 2 patients in Arm A discontinued drug due to AEs. Adverse events were generally mild and the most frequent AEs were fatigue (31.9% vs. 15.8%, P=0.015), headache (24.2% vs. 23.2%, P>0.05), and nausea (20.9% vs 6.3%, P=0.005) in Arms A and B, respectively. The proportions of patients with hemoglobin below the lower limit of normal at the end of treatment and bilirubin >3× upper limit of normal were higher in patients receiving RBV; only 1.1% (2/186) of patients experienced hemoglobin <10 g/dL, both in Arm A.

TABLE 4
Efficacy and Safety of Compound 1/r/Compound
2/Compound 3 (3D) ± RBV assessed on the
ITT and safety population, respectively, n (%)

| | Arm A | | Arm B | |
|---|---|---|---|---|
| | 3D + RBV | | 3D | |
| Efficacy | (N = 88) | | (N = 91) | |
| SVR₁₂ | 85 | (96.6) | 91 | (100) |
| On-treatment virologic failure | 0 | (0) | 0 | (0) |
| Relapse by post-treatment Week 12 | 0 | (0) | 0 | (0) |
| Study drug discontinuation | 2 | (2.3) | 0 | (0) |
| Missing SVR₁₂ data | 1 | (1.1) | 0 | (0) |
| Safety | (N = 91) | | (N = 95) | |
| Treatment-emergent AEs | 72 | (79.1) | 74 | (77.9) |
| Serious AEs | 2 | (2.2) | 2 | (2.1) |
| AEs leading to drug discontinuation | 2 | (2.2) | 0 | (0) |
| Laboratory abnormalities of interest | | | | |
| Hemoglobin decrease to below LLN^{a} | 38 | (42.0)*** | 5 | (5.5) |
| Total bilirubin >3X ULN | 8 | (8.8)** | 0 | (0) |
| Alanine aminotransferase >5X ULN | 0 | (0) | 0 | (0) |

^{a}Secondary efficacy endpoint, thus using the ITT population, N's = 88 and 91 for Arm A and B, respectively.
RBV, ribavirin; SVR₁₂, 12-week sustained virologic response; AEs, adverse events; LLN, lower limit of normal; ULN, upper limit of normal.
** and *** denote statistical significance at the .01 and .001 levels, respectively, using Fisher's exact test.

This study shows that a 12-week regimen of Compound 1/r, Compound 2 and Compound 3 with or without RBV achieved high rates of SVR12 (96.6% with RBV, and 100% with ribavirin) and was generally well tolerated, as evidenced by the low rate of treatment discontinuation and serious adverse events. The regimen without RBV was associated with lower rates of laboratory abnormalities including bilirubin elevation and hemoglobin decrease.

### Example 6. Clinical Study of Interferon-Free Treatment of HCV Genotype 1a

HCV genotype 1a-infected, treatment-naïve patients in this study were randomized 1:2 to receive either blinded ribavirin twice daily at a dose of 1000 to 1200 mg per day according to body weight (1000 mg if body weight was <75 kg, 1200 mg if body weight was ≥75 kg) (Group A) or matching placebo (Group B) for 12 weeks. All patients received open-label Compound 1/r/Compound 2 (150 mg/100 mg/25 mg once daily) and Compound 3 (250 mg twice daily) for 12 weeks. Patients were followed for 48 weeks after the treatment period. A total of 305 patients were randomized and received at least one dose of study drug. Baseline demographics and characteristics were representative of typical North American or European GT 1a-infected HCV populations. All patients were non-cirrhotic.

After 12 weeks of treatment with Compound 1/r, Compound 2 and Compound 3, the sustained virologic response rate 12 weeks after treatment (SVR12) was 97.0% (97/100) in Group A, and 90.2% in Group B. SVR12 rates for Group A and Group B were both noninferior and superior to the historical rate for telaprevir plus peginterferon/ribavirin in treatment-naïve HCV genotype 1a-infected adults without cirrhosis.

The test for heterogeneity did not show a significant difference in SVR for sex, Hispanic or Latino ethnicity, age, fibrosis, viral load and IL28B genotype. SVR12 rates of at least 95% for both treatment arms were observed in certain subgroups, including patients with IL28B CC genotype (100% in Group A vs. 97% in Group B) and female patients (100% in Group A vs. 95% in Group B). Treatment differences between Group A and Group B did not vary significantly among the subgroups evaluated.

### Example 7. Clinical Study of Interferon-Free Treatment of HCV Genotype 1

In this study, patients with Child-Pugh A cirrhosis were treated with Compound 1/r/Compound 2 (150 mg/100 mg/25 mg once daily), Compound 3 (250 mg twice daily), and weight-based ribavirin for 12 weeks. The primary efficacy analysis was the proportion of subjects achieving SVR12 compared to the historic telaprevir-based thresholds of 43% (non-inferiority) and 54% (superiority).

Eligible patients were adults 18 to 70 years old with chronic HCV genotype 1 infection and plasma HCV RNA level >10,000 IU/mL who were treatment-naïve or previously treated with peginterferon/ribavirin. All patients had cirrhosis, documented using liver biopsy or FibroScan, defined as compensated by a Child-Pugh class A score of <7 at screening, and no current or past clinical evidence of Child-Pugh B or C classification.

Patients were stratified as treatment-experienced or treatment-naïve according to previous treatment with peginterferon/ribavirin. Treatment-experienced patients were stratified by HCV subtype and by type of non-response to previous peginterferon/ribavirin treatment: null-responder, partial responder, or relapser. During the treatment period, patients received co-formulated Compound 1/r/Compound 2 (150 mg/100 mg/25 mg once daily), together with Compound 3 (250 mg twice daily) and ribavirin (1000 mg to 1200 mg divided twice daily, according to body weight), for 12 weeks.

After 12-week treatment according to the above-described regimen, the SVR12 rate was 91.8% (191 patients achieved SVR12 among a total of 208 patients studied). Table 5 summarizes the SVR12 rates among different patient populations. The SVR12 rate was noninferior and superior to the historic telaprevir plus peginterferon/ribavirin thresholds in HCV genotype 1 infected patients with cirrhosis.

At the end of the 12-week treatment, liver enzymes were normalized in most patients with baseline elevations. Activated partial thromboplastin time was normalized at the end of treatment in 47/67 (70.1%) patients with values >ULN at baseline. Mean total bilirubin values decreased to the end of treatment, and normalized post-treatment. In sum, the 12-week treatment resulted in high SVR rates and normalization of liver-related chemistry and coagulation profile abnormalities often present in patients with cirrhosis.

Model for end-stage liver disease (MELD) scores assess liver disease severity. Changes in MELD score by baseline MELD score was assessed in this study. Change in MELD score was reported for subgroups of patients with baseline MELD scores of 6-9, 10-13, or ≥14. It was determined that the combination of Compound 1/r/Compound 2 (150 mg/100 mg/25 mg once daily), Compound 3 (250 mg twice daily), and weight-based ribavirin led to high SVR12 rates and favorable safety in cirrhotic patients regardless of baseline MELD score.

TABLE 5
SVR12 Rates after 12-Week Treatment

| | Patients Achieved SVR12/ | |
|---|---|---|
| | Total Patients | |
| | (Percent) | |
| GT1a by prior treatment response | | |
| Naïve | 59/64 | (92.2%) |
| Prior null responder | 40/50 | (80.0%) |
| Prior partial responder | 11/11 | (100%) |
| Prior relapser | 14/15 | (93.3%) |
| GT1b by prior treatment response | | |
| Naïve | 22/22 | (100%) |
| Prior null responder | 25/25 | (100%) |
| Prior partial responder | 6/7 | (85.7%) |
| Prior relapser | 14/14 | (100%) |

Naïve: Never received peginterferon/ribavirin for the treatment of HCV.
Prior null responder: Received at least 12 weeks of peginterferon/ribavirin for the treatment of HCV and failed to achieve a 2 log₁₀ IU/mL reduction in HCV RNA at week 12; or received at least 4 weeks of peginterferon/ribavirin for the treatment of HCV and achieved a <1 log₁₀ IU/mL reduction in HCV RNA at Week 4 (≥25 days).
Prior partial responder: Received at least 20 weeks of peginterferon/ribavirin for the treatment of HCV and achieved ≥2 log₁₀ reduction in HCV RNA at week 12, but failed to achieve HCV RNA undetectable at the end of treatment.
Prior relapser: Received at least 36 weeks of peginterferon/ribavirin for the treatment of HCV and was undetectable at or after the end of treatment, but HCV RNA was detectable within 52 weeks of treatment follow-up.

### Example 8. Clinical Study of Interferon-Free Treatment of HCV Genotype 1

In this randomized, double-blind, placebo-controlled, multicenter trial, 631 treatment-naïve, non-cirrhotic HCV genotype 1-infected patients were assigned (3:1) to active regimen (Arm A; 473 patients) or matching placebos (Arm B; 158 patients). Arm A included administration of co-formulated Compound 1/r/Compound 2 (150 mg/100 mg/25 mg once daily), together with Compound 3 (250 mg twice daily) and weight-based ribavirin (1000 mg daily if body weight was <75 kg, 1200 mg daily if body weight was ≥75 kg), during a 12-week double-blind period. Arm B patients received matching placebos during this period. Ribavirin dose was modified due to adverse events in 5.5% of Arm A patients.

The primary endpoint was sustained virologic response 12 weeks post-treatment (SVR12). The primary analysis compared the response rate for Arm A with a historical control response rate for non-cirrhotic treatment-naïve patients who received telaprevir and peginterferon/ribavirin. Randomization was stratified by HCV subtype (1a, non-1a) and IL28B genotype (CC, non-CC).

The modified intention-to-treat SVR12 rate was 96.2% for Arm A (455 patients among the total of 473 Arm A patients achieved SVR12). This rate was noninferior and superior to the historical control SVR rate for telaprevir plus peginterferon/ribavirin. The SVR12 rate was 95.3% (307/322) in patients infected with HCV genotype 1a and 98.0% (148/151) in patients infected with HCV genotype 1b. These rates were superior to the historical control SVR rates for the respective subgroups. SVR12 rates were similarly high regardless of characteristics including IL28B genotype (CC: 96.5%, non-CC: 96.0%), race (Black: 96.4%, non-Black: 96.2%), baseline fibrosis score (F0-F1: 97.0%, F2: 94.3%, ≥F3: 92.5%), or baseline HCV RNA level (<800,000 IU/mL: 98.1%, ≥800,000 IU/mL: 95.7%). The SVR12 rate in patients with ribavirin dose modification was 93.5% (29/31) versus 96.4% (426/442) in those without modification. Even among patients with body-mass index ≥30 kg/m², the SVR12 rate was high (91.5%).

### Example 9. Clinical Study of Interferon-Free Treatment of HCV Genotype 1

In this phase 3 clinical study, 394 patients were randomized (3:1) to active regimen or placebo during a 12-week double-blind period. The randomization schedule was stratified by type of response to previous peginterferon/ribavirin treatment (relapse, partial response, or null-response) and HCV subgenotype (1a, non-1a). During the double-blind period, patients randomized to active regimen received oral co-formulated Compound 1/r/Compound 2 (150 mg/100 mg/25 mg once daily), together with Compound 3 (250 mg twice daily) and weight-based ribavirin (1000 mg daily if body weight was <75 kg, 1200 mg daily if body weight was ≥75 kg; both divided twice daily), for 12 weeks. Patients randomized to placebo received matching placebo pills during this period. Treatment assignment was blinded to the investigator, patient, and sponsor during the double-blind period. All patients enrolled in the study were non-cirrhotic, peginterferon/ribavirin dual therapy-experienced, HCV genotype 1-infected patients with prior relapse (HCV RNA undetectable at end of treatment, but detectable thereafter), or partial (≥2 log₁₀ IU/mL HCV RNA reduction at treatment week 12 but detectable at end of treatment) or null-response (<2 log₁₀ IU/mL or <1 log₁₀ IU/mL HCV RNA reduction at treatment week 12 or 4, respectively).

The primary endpoint was sustained virologic response 12 weeks post-treatment (SVR12). The primary efficacy analysis compared this rate in active regimen recipients to a historical response rate in HCV genotype 1-infected, non-cirrhotic, treatment-experienced patients who received telaprevir and peginterferon/ribavirin.

Among patients on active regimen, the SVR12 rate was 96.3% (286 of 297 patients on active regimen achieved SVR12). This was noninferior and superior to the historical control SVR rate for telaprevir and peginterferon/ribavirin. SVR12 rates among HCV-infected patients with HCV subtype 1a and 1b were 96.0% (166/173) and 96.7% (119/123), respectively. HCV subtype could not be determined for one patient, who achieved SVR12. The SVR12 rates were 95.3% (82/86) among prior relapsers, 100% (65/65) among partial responders, and 95.2% (139/146) among null-responders. SVR12 rates were also high across subgroups differing in characteristics including race, age, fibrosis score, and IL28B genotype.

Seven of the 293 patients (2.4%) experienced post-treatment viral relapse. At the time of relapse, 6 of the 7 patients had at least one variant known to confer resistance to one of the three direct-acting antivirals included in the regimen. The most frequently detected variants in the 5 genotype 1a-infected patients at the time of virologic failure were D168V (2/5) in NS3, M28V (3/5) and Q30R (2/5) in NS5A, and S556G (2/5) in NS5B. At the time of virologic failure, one of the genotype 1b-infected patients had no resistance-associated variants in NS3, NS5A or NS5B; the other genotype 1b-infected patient had Y56H and D168A in NS3, Y93H in NS5A and C316N+S556G in NS5B.

### Example 10. Clinical Study of Interferon-Free Treatment of HCV Genotype 2

In this study, 37 non-cirrhotic, peginterferon/ribavirin (pegIFN/RBV) treatment-experienced Japanese adults with chronic HCV GT2 infection were treated with Compound 1/r (100 mg/100 mg or 150 mg/100 mg; QD) and Compound 2 (QD) for 12 weeks. These treatment-experienced patients included null responders, partial responders, and/or relapsers.

The SVR12 and SVR24 rates for the Compound 1/r (100 mg/100 mg) arm were 57.9% (N=19), and for the Compound 1/r (150 mg/100 mg) arm were 72.2% (N=18). Two of 8 GT2b-infected patients treated with Compound 1/r (100 mg/100 mg) plus Compound 2 achieved SVR24; three of 8 GT2b-infected patients treated with Compound 1/r (150 mg/100 mg) plus Compound 2 achieved SVR24; nine of 11 GT non-2b-infected patients treated with Compound 1/r (100 mg/100 mg) plus Compound 2 achieved SVR24; and all ten GT2b-infected patients treated with Compound 1/r (150 mg/100 mg) plus Compound 2 achieved SVR24.

### Example 11. Clinical Study of HCV GT1 Infected Patients Receiving Chronic Opioid 1 Therapy

Non-cirrhotic patients with chronic HCV GT1 infection who were on stable methadone or buprenorphine+/−naloxone therapy were enrolled in this open-label study. Patients were treated for 12 weeks with co-formulated Compound 1/r/Compound 2 (2 tabs QD), Compound 3 (1 tab BID), and weight-based RBV (3D+RBV). The percentage of patients achieving SVR12 (HCV RNA <LLOQ 12 weeks post-treatment) was assessed in an intent-to-treat analysis.

38 patients were enrolled (19 on methadone, 19 on buprenorphine). Mean age was 48.2 years, 66% were male, 95% were treatment-naïve, 84% had GT1a infection, and 68% had IL28b non-CC genotype. One patient prematurely discontinued due to serious adverse events unrelated to study drug (cerebrovascular accident and sarcoma). The remaining 37 subjects (97.4%) all achieved SVR12. There were no virologic failures. The most frequent adverse events were nausea (50%), fatigue (47.4%), and headache (31.6%); 8 patients experienced hemoglobin <10 g/dL while on treatment, which was managed with RBV dose reduction. No dose adjustments of methadone or buprenorphine were reported. Among patients on stable methadone or buprenorphine therapy, the 3D+RBV regimen was well tolerated and achieved an SVR12 rate of 97.4%.

Another study also showed that the 3D regimen with or without RBV was well tolerated in patients on chronic opioid substitution treatment with methadone or buprenorphine, with a high SVR12 rate of over 95%.

### Example 12. Clinical Study of Patients Co-Infected with Hepatitis C and HIV-1

This was a randomized, open-label study evaluating the 3D+RBV regimen for 12 weeks. Study eligibility included: HCV treatment-naïve or pegIFN/RBV-experienced, presence or absence of cirrhosis (Child-Pugh A), CD4+ count ≥200 cells/mm³ or CD4+ % >14%, and plasma HIV-1 RNA suppressed on a stable atazanavir- or raltegravir-inclusive antiretroviral regimen. The primary endpoint is SVR 12 weeks post-treatment (SVR12). The baseline characteristics of the patients are summarized in Table 6.

Virologic response at end-of-treatment (EOTR) and 4 weeks post-treatment (SVR4) was achieved by 30/31 (96.8%) and 29/31 (93.5%) patients, respectively. One patient withdrew consent prior to finishing treatment but had an undetectable HCV RNA at last study visit (week 10), and another patient experienced virologic relapse at post-treatment week 2. No patient experienced a serious AE or discontinued study drugs due to an AE. Elevation in total bilirubin was the most common laboratory abnormality, predominantly in patients receiving atazanavir. HIV-1 RNA suppression <200 copies/mL was maintained in all patients.

The high virologic response rate and low rate of treatment discontinuation observed with 3D+RBV in treatment-naïve and treatment-experienced GT1 HCV/HIV-1 co-infected patients with or without cirrhosis is consistent with those in HCV GT1-monoinfected populations receiving this regimen.

TABLE 6
Patients Baseline Profiles

| Baseline Demographics and | 12-Week 3D + RBV | |
|---|---|---|
| Characteristics, n (%) | N = 31 | |
| Age (yrs), mean (range) | 50.9 | (38-66) |
| Sex, Male | 29 | (93.5) |
| Race, Black | 7 | (22.6) |
| HCV GT1a | 27 | (87.1) |
| IL28B Non-CC | 26 | (83.9) |
| Prior Treatment Experience | | |
| Naïve | 20 | (64.5) |
| Relapser | 1 | (3.2) |
| Partial Responder | 5 | (16.1) |
| Null Responder | 5 | (16.1) |
| Cirrhosis | 6 | (19.4) |
| HIV-1 ART Regimen | | |
| Atazanavir | 16 | (51.6) |
| Raltegravir | 15 | (48.4) |

### Example 13. 12-Week Ribavirin-Free Regimen of Ombitasvir/Paritaprevir/r and Dasabuvir for Patients with HCV Genotype 1b and Cirrhosis

Treatment with the 3 direct-acting antiviral (3D) regimen of ombitasvir, paritaprevir (boosted with ritonavir), and dasabuvir without ribavirin (RBV) for 12 weeks has demonstrated 12-week sustained virologic response (SVR12) rates of 100% in HCV genotype (GT) 1b patients without cirrhosis, and 99% in GT1b patients with compensated cirrhosis when co-administered with RBV for 12 weeks. This Example describes the safety and efficacy of the 3D regimen without RBV in patients with HCV GT1b infection and compensated cirrhosis.

Patients enrolled in this phase 3b, multicenter, open-label study received 12 weeks of 3D without RBV. Both treatment-naïve and peginterferon/RBV treatment-experienced patients with compensated cirrhosis with no history of decompensation were enrolled with the following criteria: hemoglobin ≥10 g/dL, albumin ≥2.8 g/dL, platelet count ≥25×10⁹/L, and creatinine clearance ≥30 ml/min. Efficacy was assessed by the percentage of patients achieving SVR (HCV RNA below the level of quantitation [LLOQ; <25 IU/mL]) at post-treatment week 12 (SVR12). Efficacy and safety were assessed in all patients receiving study drug.

Sixty GT1b-infected patients with compensated cirrhosis received 3D. The study population comprised 33 (55%) treatment-experienced, 50 (83%) with IL28B non-CC genotype, 13 (22%) with platelet count <90×10⁹/L, and 10 (17%) with albumin <3.5 g/dL. Serum HCV RNA decline was rapid with 37/60 (62%) patients <LLOQ at treatment week 2, and 60/60 (100%) patients <LLOQ by week 4 through end of treatment. There were no premature treatment discontinuations. All 60 patients completed treatment and 60/60 (100%) have achieved SVR12. Mean albumin levels improved from 3.9 g/dL at baseline to 4.1 g/dL by post-treatment week 4. The majority of adverse events (AEs) were mild or moderate with diarrhea (17%), headache (15%), and fatigue (12%) as the most common AEs. No clinically significant laboratory abnormalities were observed.

This study confirms that the 3D regimen without RBV for 12 weeks is well tolerated and highly efficacious in HCV GT1b-infected patients with compensated cirrhosis, including treatment-experienced patients. This study also confirms that ribavirin is not required with ombitasvir, paritaprevir (boosted with ritonavir) and dasabuvir in the treatment of HCV GT1b patients with cirrhosis.

The present invention further contemplates that in any method or treatment regimen of the invention, the patient (e.g., infected with GT1 or GT4) can have Child-Pugh B (CPB) cirrhosis. The present invention further contemplates that in any method or treatment regimen of the invention, the patient (e.g., infected with GT1 or GT4) can have decompensated cirrhosis. Studies conducted on patients with CPB cirrhosis or decompensated cirrhosis showed that 3D+RBV can effectively suppress the HCV viral level to undetectable after 8 weeks treatment.

### Example 14. Efficacy and Safety of Ombitasvir/Paritaprevir/Ritonavir Co-Administered with Ribavirin in Adults with Genotype 4 Chronic Hepatitis C Infection and Cirrhosis

HCV genotype 4 (GT4) represents approximately 20% of global HCV infection. Although GT4 infection is more common in the Middle East and sub-Saharan Africa, with globalization, GT4 is now seen increasingly in Europe and many other countries. In the Phase 2b PEARL-I study, the efficacy and safety of the two direct acting antiviral agents (2DAA) ombitasvir (OBV), a NS5A inhibitor and paritaprevir, a NS3/4A protease inhibitor co-dosed with ritonavir (PTV/r) with or without ribavirin (RBV) were assessed in 135 subjects with HCV GT4 infection without cirrhosis. SVR12 was 100% in both treatment naïve (TN) and prior interferon (IFN) and RBV treatment experienced (TE) subjects receiving 2DAA+RBV for 12 weeks. This Example extends those observations by evaluating the efficacy and safety of co-formulated OBV/PTV/r with RBV in HCV GT4-infected subjects with compensated cirrhosis.

This ongoing Phase 3, randomized, open-label, multinational study (NCT 02265237) enrolled HCV GT4-infected TN subjects or IFN/RBV or pegIFN/RBV TE subjects with compensated cirrhosis. Subjects were randomized 1:1 to receive co-formulated OBV/PTV/r co-administered with weight based RBV for 12 (Arm A) or 16 weeks (Arm B) with an approximately equal number of TN and TE subjects in each arm. A 24 week treatment arm (C) and an exploratory assessment in subjects who have experienced virologic failure with either sofosbuvir/pegIFN/RBV or sofosbuvir/RBV will follow. The primary objectives are to assess safety and SVR12 rates of these 2 DAA regimens as compared to a historical SVR12 rate for HCV GT4-infected subjects treated with pegIFN/RBV.

55 and 56 cirrhotic subjects were randomized into Arms A and B, respectively. Of the 111 subjects, 48% were TN and 52% were TE with IFN/RBV or pegIFN/RBV (30% prior nulls, 12% prior relapsers and 10% partial responders). At baseline, 91% of subjects had a Child-Pugh score of 5, 6% of 6 and 3% of 7. Overall, 72% are male, 78% White and 17% Black or African American. The mean age is 57 years and mean BMI 28 kg/m², with 29% reporting a history of diabetes. Overall DAA-related treatment emergent adverse events (AEs) occurring in ≥10% of subjects were fatigue and headache (˜15% each). 5 subjects reported a total of 12 treatment-emergent serious AEs; 1 deemed related to study drug (manic crisis). No AE led to discontinuation of study drug; 1 subject withdrew consent and 1 subject met the on-treatment criteria for virologic failure. OBV/PTV/r with RBV for 12 and 16 weeks was generally well tolerated.

In Arm A, 12 patients have been tested so far at post treatment week 4, and 12/12 (100%) have achieved SVR4. Only one virologic failure has been observed so far in Arm A. Further evaluation showed that among all patients tested so far (about 50 patients in each arm), over 95% SVR12 (ITT analysis) was achieved for both arms.

### Example 15. Efficacy and Safety of Co-Formulated Ombitasvir/Paritaprevir/Ritonavir with Ribavirin in Adults with Chronic HCV Genotype 4 Infection in Egypt

Chronic hepatitis C virus (HCV) infection is the main cause of liver cirrhosis and liver cancer in Egypt and one of the five leading causes of death. The prevalence of HCV infection in Egypt is the highest in the world (10-14%) with over 90% infected with HCV genotype (GT) 4. This Example describes the first phase 3 trial to evaluate OBV/PTV/r with RBV in Egypt for GT4 infected subjects with and without compensated cirrhosis.

This ongoing Phase 3, multicenter, open label trial enrolled 160 subjects across 5 sites in Egypt. Non-cirrhotic patients (n=100) received co-formulated OBV/PTV/r once-daily (25 mg/150 mg/100 mg) with weight based RBV for 12 weeks (Arm A). Cirrhotic subjects (n=60) were randomized 1:1 to the same regimen for either 12 or 24 weeks (Arms B and C; n=30/arm). The primary efficacy endpoint is SVR12. Safety is being evaluated by adverse event (AE) monitoring, laboratory testing, and other standard assessments. Subjects will be followed for 48 weeks post treatment.

A total of 160 noncirrhotic (Arm A, n=101) and cirrhotic (Arm B, n=30, C n=29) subjects were enrolled. Approximately half were TE (61% prior nulls, 24% prior relapsers and 15% partial responders). Overall, 76% are male. The average age is 54 years and mean BMI 29.5 kg/m², with 18% reporting a history of diabetes and 67% with HOMA-IR scores ≥3. Overall DAA-related treatment emergent adverse events (AEs) occurring in ≥10% of subjects were fatigue (12%) and headache (15%). There was 1 subject with a serious AE (SAE) of deep venous thrombosis deemed reasonably possibly related to study drug. There were no AEs leading to discontinuation of study drug, 1 subject withdrew consent (Arm A) and two subjects met the on-treatment criteria for virologic failure (Arm B, n=1 and C, n=1). The study regimen was generally well tolerated.

In Arm A, no virologic failure has been observed so far; and for the patients who have tested at post treatment week 4 or 12, 100% of the patients have achieved SVR4 or SVR12, respectively. In Arm B, only one virologic failure has been observed so far; and for the patients who have tested at post treatment week 4 or 8, 100% of the patients have achieved SVR4 or SVR8, respectively.

### Example 16. Ombitasvir/Paritaprevir/Ritonavir+Dasabuvir+/−Ribavirin (RBV) in Non-Cirrhotic HCV Genotype 1-Infected Patients with Severe Renal Impairment or End-Stage Renal Disease

HCV is common among patients with end-stage renal disease. Co-formulated ombitasvir/paritaprevir/ritonavir (25/150/100 mg QD) plus dasabuvir (250 mg BID), referred herein as “3D”, do not require dose adjustment in patients with renal insufficiency. In Phase 3 trials, 3D+/−RBV showed high SVR rates and low rates of discontinuation due to adverse events in HCV genotype 1 (GT1)-infected patients. This Example describes an open-label study evaluating 3D+/−RBV in patients with stage 4 or 5 chronic kidney disease (CKD) and GT1 infection.

Cohort 1 enrolled treatment-naïve, non-cirrhotic adults with GT1 infection and CKD stage 4 (estimated GFR 15-30 mL/min/1.73 m²) or 5 (eGFR <15 mL/min/1.73 m² or requiring dialysis). Patients received 12 weeks of 3D+RBV (GT1a) or 3D (GT1b). RBV was dosed at 200 mg QD for GT1a patients. Cohort 2 included cirrhotic patients.

20 patients in Cohort 1 received study drug. All patients (N=17) have achieved end of treatment (EOT) response. All 13 patients with available data at post-treatment week 4 and all 6 patients with available data at post-treatment week 12 achieved SVR. No virologic failures have been observed so far. One patient died 14 days post-treatment of cardiac causes unrelated to study drug. The majority of patients (19/20) had at least one adverse event, most of which were mild or moderate in severity. There were no study drug discontinuations. Nine of the 13 GT1a patients modified RBV dose due to hemoglobin decreases. There was one case of hemoglobin <8 g/dL. No blood transfusions were performed.

Among HCV GT1-infected patients with stage 4 or 5 CKD in this study, 3D+/−RBV has been well tolerated, with no premature treatment discontinuations. Hemoglobin decreases were managed with RBV interruption, which does not appear to affect efficacy.

For the 13 patients who have tested at post treatment week 4, all of them (100%) have achieved SVR4. For the 6 patients who have tested at post treatment week 12, all of them (100%) have achieved SVR12. Further evaluation showed that 90% (18/20) of all patients achieved SVR12.

Accordingly, the present invention contemplates that in any method or treatment regimen of the invention for treating GT 1, the patient can have CKD, such as stage 4 or 5 CKD. The present invention further contemplates that in any method or treatment regimen of the invention for treating GT 1, the patient can have severe renal impairment or end-stage renal disease.

### Example 17. Effect of Chronic Kidney Disease on the Pharmacokinetics of Ombitasvir, Paritaprevir, Ritonavir and Dasabuvir in Subjects with HCV Genotype 1 Infection

The all-oral interferon-free, 3 direct acting antiviral (3-DAA) regimen of ombitasvir (OBV)+paritaprevir coadministered with ritonavir (PTV/r)+dasabuvir (DSV)±ribavirin (RBV) was evaluated in HCV genotype (GT) 1-infected subjects with chronic kidney disease (CKD). No meaningful alterations in exposures were seen when the 3 DAAs were administered to HCV-uninfected subjects with renal impairment. The Example describes the effect of CKD Stage 4 and Stage 5 on the pharmacokinetics of OBV, PTV/r and DSV in HCV GT1-infected subjects.

Pharmacokinetic data from a Phase 2 study (N=38) in subjects with normal or mild renal impairment (subjects “without kidney disease”) were combined with preliminary data from a Phase 3b study in subjects with CKD Stage 4 (N=5) or Stage 5 on hemodialysis (N=14). In both studies subjects received OBV/PTV/r 25/150/100 mg QD and DSV 250 mg BID±RBV for 12 weeks. Pharmacokinetic parameters and steady-state exposures of the 3-DAA regimen were estimated using population pharmacokinetic models.

CKD was not a significant covariate in the population pharmacokinetic analyses, and the safety profile of the 3 DAAs was similar in subjects with or without CKD. PTV and DSV exposures were comparable (<22% difference) between subjects without kidney disease and CKD Stage 4. OBV and ritonavir exposures were about 80% and 200% higher, respectively, in CKD Stage 4 subjects in the limited number of subjects in this analysis. OBV and PTV exposures were comparable (<20% difference) between subjects without kidney disease and CKD Stage 5, while ritonavir and DSV exposures were about 33% and 37% lower, respectively. Based on the established safety and efficacy profile of the 3-DAA regimen, as well as exposure-response analysis, these differences in exposure are not believed to be clinically significant. Therefore, no dose adjustment is necessary in HCV genotype 1-infected subjects with CKD Stage 4 and 5.

### Example 18. Effect of Renal Function on the Pharmacokinetics of Ombitasvir/Paritaprevir/Ritonavir, Dasabuvir and Ribavirin in Over 2000 Subjects with HCV GT1 Infection

The Example evaluates the effect of renal function as estimated by creatinine clearance (CrCL) on the pharmacokinetics of OBV, PTV, DSV, RTV and RBV in HCV infected GT1 subjects.

Total exposure measured by area under the plasma concentration curve (AUC) was generated for OBV, PTV, DSV, RTV and RBV using population pharmacokinetic modeling by pooling data from 6 phase 3 studies and 1 phase 2 study in >2000 HCV GT1 infected subjects. All subjects received ombitasvir/paritaprevir/ritonavir 25/150/100 mg QD and dasabuvir 250 mg BID±weight based RBV. DAAs (OBV, PTV, or DSV) and RTV AUC values were available from 2093 subjects and RBV AUC values were available from 1584 subjects. The dataset included subjects with normal renal function (NF) (CrCl ≥90 mL/min, n=1495), mild renal impairment (RI) (CrCL 60-89 mL/min, n=576) and moderate RI (CrCL 30-59 mL/min, n=22). The effect of CrCL on the AUC values of each DAA, RTV and RBV was evaluated, and adjusted for any significant subject-specific covariates (at a significance level of 0.05) including, age, sex, body weight (BW), cirrhosis (CRHS) and Asian ethnicity (ASN) in multiple linear regression (MLR) analysis (R 3.2.0). CrCL was retained in the models, regardless of its statistical significance, to determine the effect, if any, on the AUC values. Using the final MLR model, AUC values were predicted for subjects with NF (CrCL=105 mL/min), mild RI (CrCL=75 mL/min) and moderate RI (CrCL=45 mL/min).

CrCL was not a statistically significant predictor of DAAs and RTV AUC values (p>0.05). Age, sex, CRHS were significant covariates for all DAAs/RTV while BW and ASN were for ombitasvir and dasabuvir. CrCL showed a significant relation with the RBVAUC values (p<0.05), which is consistent with RBV's predominant renal excretion. Age, sex, BW and CRHS were significant covariates for RBV. The DAA AUC values were comparable (≤10% difference) amongst different levels of renal function, while RBV AUC values were up to 17% higher in mild/moderate RI compared to NF.

HCV GT1-infected subjects with or without cirrhosis, mild/moderate RI did not affect DAA and RTV exposures; thus, no dose-adjustments are needed for the 3D regimen. RBV doses should be adjusted for renal impairment as recommended in its label.

### Example 19. Randomized Phase 3 Trial of Ombitasvir/Paritaprevir/Ritonavir for HCV Genotype 1b-Infected Japanese Patients with or without Cirrhosis

This Example describes a phase 3 trial evaluating efficacy and safety of a 12-week regimen of co-formulated ombitasvir (OBV)/paritaprevir (PTV)/ritonavir (r) for treatment of Japanese HCV genotype (GT) 1b-infected patients. The study includes a double-blind, placebo-controlled substudy of patients without cirrhosis and an open-label substudy of patients with compensated cirrhosis. Patients without cirrhosis were randomized 2:1 to once daily OBV/PTV/r (25 mg/150 mg/100 mg; Group A) or placebo (Group B). Patients with cirrhosis received open-label OBV/PTV/r (25 mg/150 mg/100 mg; Group C). A total of 321 patients without cirrhosis were randomized and dosed with double-blind study drug (106 received double-blind placebo and later received open-label OBV/PTV/r) and 42 patients with cirrhosis were enrolled and dosed with open-label OBV/PTV/r. In the primary efficacy population, the SVR12 rate was 94.6% (106/112; 95% confidence interval 90.5-98.8). SVR12 rates were 94.9% (204/215) in Group A, 98.1% (104/106) in Group B (open-label), and 90.5% (38/42) in Group C. Overall, virologic failure occurred in 3.0% (11/363) of patients who received OBV/PTV/r. The rate of discontinuation due to adverse events was 0-2.4% in the three patient groups receiving OBV/PTV/r. The most frequent adverse event in patients in any group was nasopharyngitis.

In this broad HCV GT1b-infected Japanese patient population with or without cirrhosis, treatment with OBV/PTV/r for 12 weeks was highly effective and demonstrated a favorable safety profile.

A phase 2, randomized, open-label trial showed the efficacy and safety of the DAAs ombitasvir (OBV) and paritaprevir (administered with low-dose ritonavir, PTV/r) for treatment of HCV GT1b infection in Japanese patients. Prior pegIFN/RBV treatment-experienced HCV GT1b-infected Japanese patients without cirrhosis received 100/100 mg or 150/100 mg PTV/r plus 25 mg OBV once daily for 12 or 24 weeks. High SVR12 and SVR24 rates (with a concordance of 100%) and a low rate of discontinuation due to adverse events were observed in HCV GT1b-infected patients regardless of treatment duration or PTV/r dose. This Example provides the efficacy and safety results from the phase 3 study, which examined the IFN- and RBV-free regimen of co-formulated OBV/PTV/r in Japanese treatment-naïve and treatment-experienced HCV GT1b-infected patients with and without cirrhosis.

This phase 3 trial included 2 substudies (1 double-blind and placebo-controlled, 1 open-label) as described above as well as below. Eligible patients were male or female, treatment-naïve or treatment-experienced (previously treated with an IFN-based therapy, such as IFN alpha, beta, or pegIFN, with or without RBV), 18-75 years old (inclusive), with chronic HCV GT1b infection and HCV RNA level >10,000 IU/ml. Patients were excluded if they were co-infected with HBV or HIV, were previously treated with a DAA, or had any cause of liver disease other than chronic HCV infection. Substudy 1 enrolled patients with no past or current clinical evidence of cirrhosis. Substudy 2 enrolled patients with compensated cirrhosis (Child-Pugh score A), no clinical history of liver decompensation, serum alpha-fetoprotein ≤100 ng/mL, and no evidence of hepatocellular carcinoma on imaging. In each Substudy, presence or absence of cirrhosis was based on liver biopsy, FibroScan, Fibrotest/APRI, or Discriminant score test.

In Substudy 1, patients without cirrhosis were randomized 2:1 to receive double-blind OBV/PTV/r 25 mg/150 mg/100 mg (Group A) or double-blind placebo (Group B) once daily for 12 weeks. Following the double-blind period, patients in Group B received 12 weeks of open-label OBV/PTV/r 25 mg/150 mg/100 mg once daily. The randomization was stratified according to prior IFN-based therapy (naïve versus experienced). Treatment-naïve patients were further stratified by HCV RNA level (<100,000 IU/ml versus ≥100,000 IU/ml). Patients with HCV RNA ≥100,000 IU/ml were further stratified by eligibility for IFN-based therapy (eligible versus ineligible). Previously IFN-treated patients were further stratified by type of previous response to IFN-based therapy (relapse, nonresponder, or intolerant to IFN-based therapy). The randomization schedule was computer-generated by the sponsor. Sites utilized interactive response technology for randomization of patients to treatment.

The investigators, patients, and sponsor were unaware of the treatment assignment during the double-blind period. To prevent implicit unblinding, investigators, patients, and sponsor were also blinded to levels of HCV RNA, IP-10, alanine aminotransferase (ALT), aspartate aminotransferase (AST), bilirubin (indirect and total), and gamma-glutamyl transferase (GGT).

In Substudy 2, patients with compensated cirrhosis were enrolled into Group C and received open-label OBV/PTV/r 25 mg/150 mg/100 mg once daily for 12 weeks.

Of 467 patients screened, 321 patients without cirrhosis were randomized in Substudy 1 (215 to double-blind OBV/PTV/r [Group A], 106 to double-blind placebo [Group B]) and 42 patients with cirrhosis were enrolled in Substudy 2 (open-label OBV/PTV/r [Group C]). Among patients with cirrhosis (Substudy 2), 78.6% were treatment-experienced, and mean (standard deviation) baseline platelet count, albumin, and international normalized ratio (PT-INR) were 114.2(47.4)×10⁹ cells/L, 38.2(3.9) g/L, and 1.060(0.091), respectively.

In the primary efficacy population, the SVR12 rate was 94.6% (106/112, 95% CI 90.5-98.8). The overall SVR12 rate among patients without cirrhosis in Group A was 94.9% (204/215); the SVR12 rates in all treatment-naïve and treatment-experienced patients were 94.2% (131/139) and 96.1% (73/76) respectively.

The overall SVR12 rate in patients without cirrhosis receiving open-label OBV/PTV/r (Group B) was 98.1% (104/106); SVR12 rates in treatment-naïve and treatment-experienced patients were 98.5% (67/68) and 97.4% (37/38) respectively in this group. The overall SVR12 rate in patients with cirrhosis receiving open-label OBV/PTV/r (Group C) was 90.5% (38/42), including 100% (9/9) and 87.9% (29/33) in treatment-naïve and treatment-experienced patients, respectively. SVR12 rates for all other predefined subpopulations were greater than 90% (see Table 7, 95% CIs were calculated using Wilson score method).

TABLE 7
SVR12 Rates in Subpopulations of Patients Without Cirrhosis

| | Group A | | | Group B | | |
|---|---|---|---|---|---|---|
| | N = 215 | | | N = 106 | | |
| | n/N | % (95% CI) | | n/N | % (95% CI) | |
| All patients without cirrhosis | 204/215 | 94.9 | (91.1-97.1) | 104/106 | 98.1 | (93.4-99.5) |
| Treatment-naïve | 131/139 | 94.2 | (89.1-97.1) | 67/68 | 98.5 | (92.1-99.7) |
| HCV RNA <100,000 IU/mL | 6/6 | 100 | (61.0-100) | 2/2 | 100 | (34.2-100) |
| IFN ineligible | 21/23 | 91.3 | (73.2-97.6) | 9/10 | 90.0 | (59.6-98.2) |
| Treatment-experienced | 73/76 | 96.1 | (89.0-98.6) | 37/38 | 97.4 | (86.5-99.5) |
| Relapser | 21/22 | 95.5 | (78.2-99.2) | 10/11 | 90.9 | (62.3-98.4) |
| Nonresponder | 28/28 | 100 | (87.9-100) | 14/14 | 100 | (78.5-100) |
| IFN Intolerant | 24/26 | 92.3 | (75.9-97.9) | 13/13 | 100 | (77.2-100) |

In patients without cirrhosis with ALT levels >ULN at baseline, ALT normalized at the end of the double-blind treatment period in a significantly greater proportion in patients receiving OBV/PTV/r versus placebo (94.3% [116/123] versus 18.9% [10/53]; P<0.001).

Resistance associated variants (RAVs) in NS3/4 and NS5A were detected in 1% and 38% of patients at baseline, respectively. The most commonly detected NS3A and NS5A RAVs in baseline samples were D168E (4/351, 1%) and Y93H (49/357, 14%), respectively. RAVs were observed in both NS3 and NS5A at the time of virologic failure in 10 of the 11 patients who experienced over-treatment-viral-failure or relapse. In NS3, D168V alone or in combination with Y56H was observed in 73% (8/11) of patients, D168A in combination with Y56H was observed in 2 patients, and 1 patient did not have any treatment emergent RAVs in NS3. In NS5A, Y93H was pre-existing in 8 patients and at the time of failure; Y93H alone or in combination with L28M, R30Q, L31M, L31V, and/or P58S was observed in 91% (10/11) of patients; L31F was observed in 1 patient.

Rates of treatment-emergent adverse events (TEAEs) in the three patient groups are also analyzed. During the double-blind period, a greater percentage of patients without cirrhosis receiving OBV/PTV/r than placebo experienced TEAEs (68.8% [148 of 215 patients] versus 56.6% [60 of 106 patients], P<0.05). TEAEs were predominantly Grade 1 or 2 in severity. TEAEs occurring with a frequency greater than 5% among patients without cirrhosis during the double-blind period in either treatment group were nasopharyngitis (16.7% [36 patients], OBV/PTV/r; 13.2% [14 patients], placebo), headache (8.8% [19 patients], OBV/PTVr; 9.4% [10 patients], placebo), and peripheral edema (5.1% [11 patients], OBV/PTV/r; 0%, placebo). The only TEAE significantly more frequent with OBT/PTV/r versus placebo during the double-blind period was peripheral edema. The proportions of serious TEAEs and TEAEs leading to study drug discontinuation were not significantly different in patients receiving OBV/PTV/r verus placebo (3.3% [7 patients] versus 1.9% [2 patients], P>0.05; and 0.9% [2 patients] versus 0%, P>0.05, respectively). TEAEs leading to study drug discontinuation in patients receiving OBV/PTV/r were anuria and hypotension in one patient each.

The TEAE profile in patients without cirrhosis receiving open-label OBV/PTV/r was comparable to that of patients without cirrhosis receiving double-blind OBV/PTV/r. TEAEs were predominantly Grade 1 or 2. TEAEs occurring with a frequency greater than 5% in this group were nasopharyngitis (7.5% [8 patients]) and headache (6.6% [7 patients]). Peripheral edema occurred in 3.8% (4 patients) of patients. Serious TEAEs occurred in 2.8% (3 patients) of patients in this group, and no patient discontinued treatment due to TEAEs.

Among patients with cirrhosis receiving open-label OBV/PTV/r, 73.8% (31 of 42 patients) experienced at least 1 TEAE. TEAEs were predominantly Grade 1 or 2 in severity. TEAEs occurring with a frequency greater than 5% were nasopharyngitis (14.3% [6 patients]), pyrexia (9.5% [4 patients]), nausea (7.1% [3 patients]), peripheral edema (7.1% [3 patients]), decreased platelet count (7.1% [3 patients]), and headache (7.1% [3 patients]). Serious TEAEs occurred in 4.8% (2 patients) of patients with cirrhosis. One patient (2.4%) had a serious TEAE (pulmonary edema) that led to study drug discontinuation.

All patients in the study who experienced a TEAE of peripheral edema were using concomitant calcium channel blockers (CCBs). Additional analyses indicated that the incidence of any edema-related TEAEs (defined as peripheral edema, edema, face edema, or pulmonary edema) was related to the use and dose of CCBs.

There were no hemoglobin decreases <8 g/dL. No patient received erythropoietin or blood transfusions during the study. No patient had a decrease in platelet count below 50×10⁹/L.

The results from this phase 3 trial in Japanese patients with HCV GT1b infection with or without cirrhosis confirmed that high SVR rates can be achieved with 12 weeks of the IFN-free and RBV-free regimen of OBV/PTV/r. High SVR12 rates were achieved with the IFN- and RBV-free OBV/PTV/r regimen in HCV GT1b-infected Japanese patients. This 2-DAA regimen was well-tolerated with low rates of discontinuation due to TEAEs.

### Example 20. Effect of Food on Bioavailability of Ombitasvir/Paritaprevir/Ritonavir (OBV/PTV/r) Co-Formulated Tablets in Healthy Japanese Subjects

In Western subjects, relative to fasting conditions, administration of ombitasvir, paritaprevir and ritonavir with a moderate fat or high fat meal increased the mean AUC by 76% to 82%, 180% to 211%, 44% to 49%, respectively. This Example studies Japanese healthy volunteers to evaluate the effect of food on the bioavailability of ombitasvir/paritaprevir/ritonavir co-formulated tablets.

Japanese male and female volunteers 20 to 55 years of age in general good health with a body mass index ≥18.5 and <25 kg/m² were eligible to enroll. Subjects who had positive test results for hepatitis A, B, or C, or for HIV infection, and subjects who were using known inhibitors or inducers of CYP3A isozyme or organic anion transporting polypeptide 1B1 (OATP1B1) inhibitors were excluded from participation. Subjects were not to have consumed alcohol, grapefruit, star fruit, or Seville oranges within 72 hours, or to have used nicotine-containing products within 6 months before study drug administration.

This was a 2-sequence 2-period crossover study. A single dose of two ombitasvir/paritaprevir/ritonavir 12.5/75/50 mg coformulated tablets (total dose of 25/150/100 mg) was administered in the morning on Study Day 1 of each period as follows:

| | |
|---|---|
| Regimen A | Under fasting conditions. |
| Regimen B | Under non-fasting conditions with a high-fat breakfast |
| | (~900 Kcal, with 35% calories from fat) 30 minutes prior |
| | to dosing |

PK parameters for ombitasvir, paritaprevir, and ritonavir were estimated by noncompartmental methods using Phoenix WinNonlin, version 6.3 (Pharsight, A Certara® Company, St. Louis, Mo.) including maximum plasma concentration (Cₘₐₓ), time to Cₘₐₓ (Tₘₐₓ), area under the plasma concentration-time curve (AUC), and terminal-phase elimination half-life (t_{1/2}). The effect of food on the bioavailability of ombitasvir, paritaprevir, and ritonavir was assessed using a repeated measures analysis of natural logarithms of Cₘₐₓ and AUC values. Point estimates of central value ratios and their 90% confidence intervals (CIs) for Cₘₐₓ and AUC were calculated to quantify the magnitude of food effect. Statistical analyses were conducted using SAS, version 9.2 (SAS Institute, Inc., Cary, N.C.).

20 male subjects in Japan were enrolled, with mean age of 28.9 years (ranging from 20 to 45 years) and mean weight of 63.3 kg (ranging from 51 to 78 kg). In these Japanese subjects, relative to fasting conditions, administration of ombitasvir, paritaprevir and ritonavir with a high fat meal increased delayed the mean Tₘₐₓ of ombitasvir, paritaprevir, and ritonavir by ˜1 h (from 4.4 to 5.3 h), ˜1 h (from 4.2 to 5.2 h), and ˜1.5 h (from 3.4 to 4.9 h), respectively, and increased the mean AUC of ombitasvir, paritaprevir, and ritonavir by 73%, 228%, and 34%, respectively.

The regimens tested were generally well tolerated by the subjects in this study. No clinically significant vital signs, ECG, laboratory measurements or physical findings were observed during the course of the study. There were no serious adverse events or discontinuations due to adverse events during the study.

The study demonstrated that a high-fat breakfast increased the bioavailability of ombitasvir/paritaprevir/ritonavir co-formulated tablets in Japanese subjects. The magnitude of increase in bioavailability observed in Japanese subjects is similar to the food effect previously observed in Western subjects following a moderate-fat or high-fat breakfast. As a result, in Japanese subjects, the ombitasvir/paritaprevir/ritonavir co-formulated tablets should be taken with food, the same as in Western subjects.

### Example 21. Bioequivalence Assessment of Ribavirin Tablets: A Randomized, Single-Dose, Open-Label, Two-Period Crossover Study in Healthy Volunteers

Ribavirin is a nucleoside analogue with antiviral activity. Ribavirin has shown both in vitro and in vivo activity against a wide range of RNA and DNA viruses, including the hepatitis C virus. The mechanism of action (MOA) by which ribavirin inhibits HCV is not fully understood. The MOA may include direct inhibition of HCV replication, inhibition of inosine monophosphate dehydrogenase, induction of mutagenesis, and/or enhancement of the immune response. Ribavirin alone has a limited effect on HCV RNA levels or on improving hepatic histology, however has shown to be effective in combination with other agents for the treatment of chronic hepatitis C.

Ribavirin is extensively absorbed with an absolute bioavailability of approximately 50%. There is a linear relationship between dose and area under the concentration-time curve (AUC) following single doses of 200 to 1,200 mg. The dose-maximum drug concentration (Cₘₐₓ) relationship is curvilinear, tending to asymptote above single doses of 800 mg.

Ribavirin is available in the US under the Copegus® and Rebetol® brand name as 200 mg tablets as well as under Ribasphere® and Moderiba® brand names as 200 mg, 400 mg and 600 mg tablets. Available anti-HCV regimens for adults typically require 800-1400 mg of ribavirin per day, administered twice daily in divided doses. For Ribasphere® and Moderiba®, tablets of all three strengths are proportionally equivalent in their active and inactive ingredients. The formulations vary only in the composition of the non-functional film coating. In vitro dissolution tests show all 3 strengths have similar, rapid release of ribavirin. Therefore, the bioequivalence assessment was conducted at the highest dose strength (600 mg) of Ribasphere.

The objective of this bioequivalence study was to compare the bioavailability of two ribavirin tablet products—600 mg Ribasphere tablets manufactured by Kadmon/DSM Pharmaceuticals (Test) and 200 mg Copegus tablets sold by Roche (Reference).

Phase 1, single-dose, non-fasting, open-label, two-period, randomized crossover study was used. 12 subjects in Group I were each dosed with a single Ribasphere tablet (600 mg ribavirin) on the morning of Day 1 after the start of a moderate fat breakfast; and 12 subjects in Group II were each dosed with three Copegus tablets (3×200 mg ribavirin) on the morning of Day 1 after the start of a moderate fat breakfast. After a 42-day washout period, each patient in Group 1 was dosed with three Copegus tablets (3×200 mg ribavirin), and each subject in Group 2 was dosed with Ribasphere tablet (600 mg ribavirin). Intensive blood samples for pharmacokinetic assessment were collected up to 72-hours after each dose.

Plasma concentrations of ribavirin were determined using a validated liquid-liquid extraction HPLC method with tandem mass spectrometric detection. Pharmacokinetic parameter values of ribavirin were estimated using non-compartmental methods.

A linear mixed effects analysis including effects for sequence, period, and regimen was performed on the natural logarithms of Cₘₐₓ, AUCₜ, and AUC_{inf}. Relative bioavailability of the regimens was assessed by a two one-sided tests procedure via 90% confidence interval for the difference of the least squares means. Bioequivalence between regimens was concluded if the antilogarithm of the 90% confidence intervals were within the 0.80 to 1.25 range.

Safety and tolerability were assessed throughout the study, including adverse events, physical examinations, brief neurological examination, vital signs, ECGs and clinical laboratory tests.

Ribavirin demonstrated a long terminal half-life and low intrasubject variability. Pharmacokinetic sampling for 72-hours after study drug administration was sufficient to ensure complete gastrointestinal transit of the solid dosage forms.

For one Ribasphere tablet (600 mg ribavirin), the 90% confidence intervals for Cₘₐₓ, AUCₜ and AUC_{inf} central value ratios fell within the bioequivalence range (0.80, 1.25), relative to three Copegus tablets (3×200 mg ribavirin). All AEs were mild in severity and assessed by the investigator as having no reasonable possibility of being related to Ribasphere or Copegus tablets. No deaths, serious AEs, subject discontinuations, or clinically significant abnormal vital signs, ECG, or laboratory measures were observed in the study.

This study showed that Ribasphere 600 mg tablet was bioequivalent to three Copegus 200 mg tablets.

### Example 23. Retreatment of HCV Genotype 1 DAA-Failures with Ombitasvir/Paritaprevir/r, Dasabuvir, and Sofosbuvir

Retreatment options for HCV patients who fail treatment with direct-acting antiviral (DAA) regimens was not yet clearly defined. Resistance-associated variants in NS5A have been shown to persist up to 96 weeks post-treatment. This study evaluated the safety and efficacy of ombitasvir/paritaprevir/ritonavir (OBV/PTV/r) and dasabuvir (DSV) plus sofosbuvir (SOF) in DAA-experienced patients with HCV genotype (GT) 1 infection.

Patients with GT1b infection without cirrhosis received OBV/PTV/r+DSV+SOF for 12 weeks; ribavirin (RBV) was administered to patients with GT1a infection without cirrhosis. GT1a-infected patients with cirrhosis received 24 weeks of OBV/PTV/r+DSV+SOF+RBV. Enrolled patients must have had history of previous DAA treatment failure without discontinuation for reasons other than virologic failure. Efficacy was assessed by SVR.

Twenty-two DAA-experienced patients were enrolled including 20 with GT1a infection and 6 with compensated cirrhosis. Prior DAAs included in the previous failed treatment regimens included OBV/PTV/r+DSV (n=14), OBV/PTV/r (n=2), telaprevir (n=2), SOF (n=2), simeprevir/samatasvir (n=1), and simeprevir+SOF (n=1). 100% SVR4 was achieved. Among the patients tested for SVR12, 93% (12/13) SVR12 was achieved for HCV GT 1a patients without cirrhosis, and 100% SVR12 (2/2) was achieved for HCV GT 1b patients. The treatment was well tolerated with no discontinuations due to treatment-related AEs and no reported treatment-related serious AEs.

This study showed that the multi-targeted regimen of OBV/PTV/r+DSV±RBV in combination with SOF is an effective retreatment strategy for patients who fail DAA-containing HCV regimens, including those containing an NS5A inhibitor.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method of treatment for a patient infected with HCV genotype 1b, comprising administering Compound 1 or a pharmaceutically acceptable salt thereof, and Compound 2 or a pharmaceutically acceptable salt thereof, to said patient, wherein said treatment does not include administration of either interferon or ribavirin to said patient, and said treatment lasts from 8 to 12 weeks, and wherein Compound 1 or the salt thereof is administered with ritonavir.

2. The method of claim 1, wherein said treatment lasts 8 weeks.

3. The method of claim 1, wherein said treatment lasts 12 weeks.

4. The method of claim 1, comprising administered 150 mg Compound 1, 100 mg ritonavir, and 25 mg Compound 2 to said patient once daily.

5. The method of claim 4, wherein Compound 1, ritonavir and Compound 2 are co-formulated in a solid dosage form.

6. The method of claim 5, wherein said patient is a treatment-naïve patient.

7. The method of claim 5, wherein said patient is an interferon null responder.

8. A method of treatment for a patient infected with HCV genotype 4, comprising administering Compound 1 or a pharmaceutically acceptable salt thereof, and Compound 2 or a pharmaceutically acceptable salt thereof, to said patient, wherein said treatment does not include administration of interferon to said patient, and said treatment lasts from 8 to 12 weeks, and wherein Compound 1 or the salt thereof is administered with ritonavir.

9. The method of claim 8, wherein said treatment lasts 8 weeks.

10. The method of claim 8, wherein said treatment lasts 12 weeks.

11. The method of claim 8, further comprising administered ribavirin to said patient.

12. The method of claim 8, wherein said treatment does not include administration of ribavirin to said patient.

13. The method of claim 8, comprising administered 150 mg Compound 1, 100 mg ritonavir, and 25 mg Compound 2 to said patient once daily.

14. The method of claim 13, wherein Compound 1, ritonavir and Compound 2 are co-formulated in a solid dosage form.

15. The method of claim 14, wherein said patient is a treatment-naïve patient.

16. The method of claim 14, wherein said patient is an interferon null responder.
